(19) United States
(12) Patent Application Publication
(10) Pub. No.: US 2013/0302640 A1
(43) Pub. Date: Nov. 14, 2013

(54) FITTING WITH ANTIBACTERIAL COATING AND METHOD FOR MANUFACTURING THE SAME

(71) Applicants: Hans-Georg Neumann, Rostock (DE); Cornelia Prinz, Marlow (DE); Ulrich Lembke, Rostock (DE)

(72) Inventors: Hans-Georg Neumann, Rostock (DE); Cornelia Prinz, Marlow (DE); Ulrich Lembke, Rostock (DE)

(21) Appl. No.: 13/996,172

(22) PCT Filed: Dec. 23, 2010
(86) PCT No.: PCT/EP10/07926

§ 371 Date: Jul. 31, 2013

(51) Int. Cl. A61L 2/238

(52) U.S. Cl. CPC A61L 2/238
USPC 428/650; 428/457; 428/336; 428/677; 427/250

(57) ABSTRACT

A fitting (10) with a substrate (12) and an antibacterial coating (14) is characterized in that the coating (14) is a PVD coating which contains copper. A method for manufacturing such fitting (10) is characterized in that the coating (14) is applied by a PVD method.

This invention relates to a fitting with an antibacterial coating and a method for manufacturing the same. A “fitting” in the sense of this application is an object which is provided for the manual operation of other objects, for example a door handle or a door knob, a window handle, a furniture fitting, a sanitary fitting, a light switch, etc.

When such fittings are used in public facilities, they are touched by many persons. There is a risk that bacteria are transmitted from the hand of a user to the fitting and from there to the next user. Therefore, various attempts have been made to provide in particular door handles, but also other fittings, with an antibacterially effective coating. One example can be found in WO 2010/036189, in which a method for electrolytically coating a door handle or similar objects is described. The door handle is electrolytically coated, in order to apply a coating containing silver.

A similar approach is pursued in DE 103 05 142 A1. Here as well, silver particles are used in a door handle, in order to produce a germicidal effect. Other than in WO 2010/036189 A1 no coating is applied here onto the door handle, but a plastic sleeve is mounted on the door handle, which contains silver particles on its surface.

A comparable construction is known from WO 2009/112173, wherein instead of a sleeve a cover of knit or woven fabric is used here, which can be mounted on the door handle. Silver threads are woven into the fabric. As alternative materials with antibacterial effect copper, gold and titanium are also mentioned.

The use of separate sleeves or covers which are provided with antibacterially effective materials is not satisfactory both with regard to the optical appearance and the handling.

The high antibacterial effectiveness of silver is generally known. However, it is also known from literature that silver ions also have an cytotoxic effect. This effect already starts at concentrations of greater than 380 μg/l (see for example Heidenau, F., Mittelmeier W., Detsch R., Haenle M., Stenzel F., Zeigler G., Gollwitzer H., 2005: “A novel antibacterial titania coating: Metall ion toxicity and in vitro surface colonization”, J. Materials Science, Materials in Medicine 16, 1-6.

The cytotoxicity of silver ions observed already at such low concentrations corresponds with the fact that silver does not play a role in the metabolism of the cell; it does not belong to the so-called essential trace elements in human organism. Taking low doses of silver and also a release from silver surfaces over an extended period can lead to permanent cell damages. WHO recommends not to consume more than 180 μg of silver per day (see for example Gibbins, B., Warner, L., 2005: “The Role of Antimicrobial Silver Nanotechnology”, MDDI, http://www.devicelink.com/mddi/archive/05/08/005.html, 05.02.08)

It is also known that copper has an antibacterial effect, which however is lower than that of silver. Correspondingly, U.S. Pat. No. 5,958,440 describes antibacterial coatings with e.g. copper. These coatings are applied using PVD methods (physical vapor deposition; physical gas phase separation). However, there are only obtained layers with a limited hardness, which are not suitable for some applications.

To utilize the antibacterial effect of copper, door handles also are known from the prior art, which are solidly made of a copper alloy. Such door handles, however, are not competitive with regard to the manufacturing costs and the necessary maintenance effort.

The object of the invention consists in creating a fitting which can be manufactured at low cost, has good antibacterial properties and in terms of optical appearance and handleability does not differ from conventional fittings without antibacterial effect. The object of the invention furthermore consists in creating a method for manufacturing such fitting.

For the solution of this object, a fitting with a substrate and an antibacterial coating is provided in accordance with the invention, which is characterized in that the coating is a PVD coating which contains copper. The invention is based on the finding that the antibacterial effect can be achieved already with a very thin layer (and correspondingly little use of copper). At the same time, a PVD coating provides for a very large hardness and stability of the coating, so that a long useful life is ensured.

As further constituents of the coating, the metals nickel, zinc, tin and aluminum can be contained individually or in any combination. The copper content in the coating always is above 50%, in order to ensure a sufficient antibacterial effect.

The coating in particular can consist of copper and nickel. The use of nickel offers the advantage that a silver-colored coating can be achieved, which optically does not differ from known fittings which are made of brushed aluminum or brushed stainless steel. This is of great advantage for the acceptance.

Preferably, the coating chiefly contains copper, the rest being nickel and unavoidable impurities. A copper content of more than 70% is particularly preferred. This ensures that enough copper is released, in order to have the desired antibacterial effect.

Particularly preferably, the coating consists of about 75% copper and about 25% nickel. This alloy, which is also known under the name cupro-nickel, is used for example for the shiny silver-colored ring of 2-Euro coins. Therefore, this alloy is available at competitive prices.

In a further preferred variant, the coating consists of a copper content of more than 80% and the metals aluminum, zinc and tin. With this combination it is possible to produce antibacterial coatings with a golden tint and high abrasion resistance.

What is preferred particularly is a coating which consists of 89% copper, 5% aluminum, 5% zinc and 1% tin. This alloy is also known as Nordic gold and is used e.g. for the 10 cent, 20 cent and 50 cent coins.

Preferably, it is provided that the coating has a thickness of 1 μm to 7 μm, in particular in the range from 2 μm to 4 μm. It was found that such a small layer thickness on the one hand can provide the necessary amount of copper, and on the other hand can be mounted on the substrate with the necessary adhesive strength.

As substrate, any material can be used in principle, on which a PVD coating can be applied. Steel or aluminum alloys are particularly useful, since the same correspond to the known fittings both in terms of weight and in terms of haptics (in particular due to their thermal conductivity).

The above-mentioned object also is solved by a method for manufacturing a fitting, wherein the coating is applied by a PVD method. With regard to the resulting advantages, reference is made to the above explanations.

In the coating, there can be used a source with a mixed target which contains copper. A mixed target provides for applying a second alloying constituent simultaneously with copper, with which the properties of the coating can be influenced in the desired way.

The use of a mixed target has the advantage that the amounts of the individual metals in the coating result from the composition of the target and thus are the same for all coating operations.

Preferably, a mixed target which consists of cupro-nickel is used for the coating. This provides for applying a coating in steel optics, so that the coated fitting does not differ from known fittings which are made of brushed stainless steel or aluminum.

Alternatively, there can also be used several evaporator sources with different targets. The expenditure for ensuring the composition of the layer will rise, however. This variant only is preferred when no suitable mixed targets are present.

The use of several evaporator sources with the same mixed targets will reduce the time for coating and therefore is preferred.

The invention will be described below with reference to an embodiment represented in the attached drawings, in which:

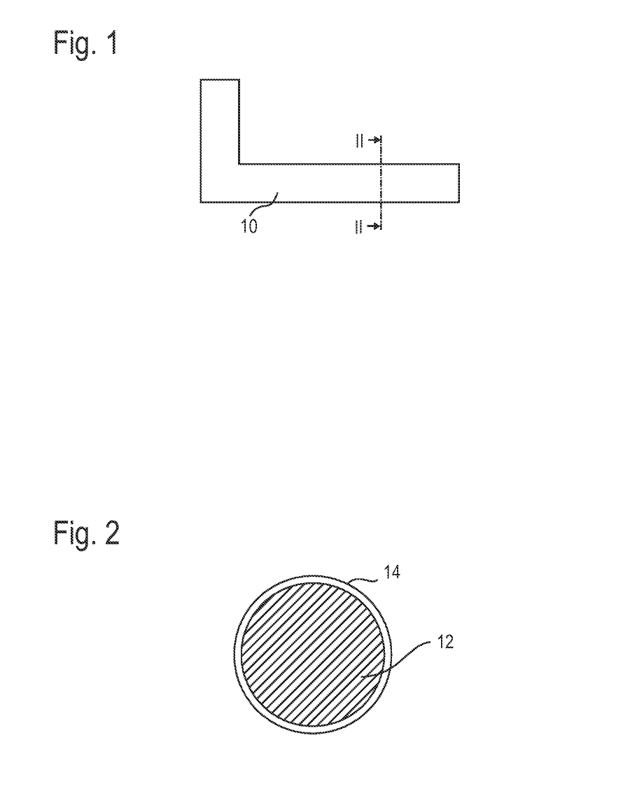

FIG. 1 schematically shows a door handle in a top view; and

FIG. 2 schematically shows a section along the plane II-II of FIG. 1.

FIG. 1 shows a door handle 10 as an example for a fitting. The fitting just as well might be a window handle, a furniture fitting, a part of a sanitary fitting, a light switch, an actuating lever of a soap dispenser or disinfectant dispenser, etc., i.e. generally an object which must be operated or actuated by a multitude of different persons.

The door handle 10 consists of a substrate 12 on which a coating 14 is applied. The substrate 12 in particular can consist of a steel or aluminum alloy. In general, there can be used any material with which fittings usually are made. In contrast to the schematic representation in FIG. 2, the substrate 12 also can be designed hollow. It is also possible that the substrate 12 is a composite component. It is only important that the outer surface of the substrate 12 is designed such that it can be coated by a PVD method.

The coating 14 is a PVD coating which in the preferred exemplary embodiment consists of an alloy which (apart from unavoidable impurities) consists of 75% copper and 25% nickel. In the PVD coating method, this material can be provided as mixed target and leads to a silver-colored coating whose tint corresponds to the silver-colored ring of 2-Euro coins. The thickness of the coating is between 1 μm and 7 μm, preferably between 2 μm and 4 μm.

In experiments it could be shown that with such a thin coating a good antibacterial effect can be achieved already:

For demonstrating the antibacterial effect of the layer according to the invention three door handles were used. There is used one door handle of pure copper and two of stainless steel. One of the door handles of stainless steel was provided with a layer of 75% copper and 25% nickel according to the invention. The coating was made in a PVD plant by means of an arc evaporator source.

All three door handles were sterilized by heating them for 1 hour at 135° C.

All the following work was carried out under sterile conditions. Now, a germ suspension was prepared with the germ Staphylococcus aureus. Subsequently, all three door handles each were drizzled with 1 ml of germ solution on the side facing upwards and thereafter dried at room temperature. This resulted in a loading of approximately 10 U+08 CFU (colony-forming units) for each door handle.

The door handles now were left in a sterile beaker for 7 hours at room temperature. Subsequently, they were cleansed with a sterile washing solution in an ultrasonic bath. The washing solution was plated on mannitol-sodium chloride agar, incubated for 24 hours at 37° C. and subsequently counted.

The following values were determined:

| | | |
|---|---|---|
| copper: | 1 | CFU |
| stainless steel with Cu/Ni coating: | 8 | CFU |
| stainless steel | 6840 | CFU |

It could be demonstrated that the coating according to the invention has a distinct antibacterial effect.

By suitable selection of the method parameters during PVD coating, the coating 14 is made such that it has a mat surface which in its surface structure and its optical appearance corresponds to brushed steel or brushed aluminum. Thus, the fitting by no means optically differs from conventional fittings.

1. A fitting with a substrate and an antibacterial coating, characterized in that the coating is a PVD coating which contains copper.

2. The fitting according to claim 1, characterized in that the coating chiefly consists of copper.

3. The fitting according to claim 2, characterized in that the coating consists of copper and nickel.

4. The fitting according to claim 3, characterized in that the coating contains a copper content of more than 50%, wherein the rest consists of nickel and unavoidable impurities.

5. The fitting according to claim 4, characterized in that the coating contains a copper content of more than 70%, wherein the rest consists of nickel and unavoidable impurities.

6. The fitting according to claim 5, characterized in that the coating consists of about 75% copper and about 25% nickel as well as unavoidable impurities.

7. The fitting according to claim 1, characterized in that beside copper the coating also contains nickel, tin and/or aluminum.

8. The fitting according to claim 7, characterized in that the coating contains at least about 80% copper as well as aluminum, zinc, tin and unavoidable impurities.

9. The fitting according to claim 8, characterized in that the coating contains about 89% copper, about 5% aluminum, about 5% zinc, about 1% tin and unavoidable impurities.

10. The fitting according to claim 1, characterized in that the coating has a thickness of 1 μm to 7 μm.

11. The fitting according to claim 1, characterized in that the coating has a thickness of 2 μm to 4 μm.

12. The fitting according to claim 1, characterized in that the fitting is a door handle, a light switch or a part of a sanitary fitting.

13. The fitting according to claim 1, characterized in that the substrate is a steel or aluminum alloy.

14. A method for manufacturing a fitting, characterized in that the coating is applied by a PVD method.

15. The method according to claim 14, characterized in that for coating there is used a source with a mixed target which contains copper.

16. The method according to claim 14, characterized in that for coating a mixed target is used, which consists of cupro-nickel.
